(19) United States
(12) Patent Application Publication
(10) Pub. No.: US 2025/0352889 A1
(43) Pub. Date: Nov. 20, 2025

(54) SYSTEMS, DEVICES, AND METHODS FOR RECORDING A TIME NOTATION FOR CORRELATION WITH A VIDEO RECORDING, AN AUDIO RECORDING, AND/OR A PHYSIOLOGICAL SIGNAL

(71) Applicant: Ryan McGowan, Huntington Beach, CA (US)

(72) Inventor: Ryan McGowan, Huntington Beach, CA (US)

(21) Appl. No.: 19/211,121

(22) Filed: May 16, 2025

Related U.S. Application Data
(60) Provisional application No. 63/649,297, filed on May 17, 2024.

(51) Int. Cl. A63F 13/216; A61B 5/024; A61B 5/11

(52) U.S. Cl. CPC A63F 13/216; A61B 5/024; A61B 5/1112; A61B 2503/12; A61B 2503/22; A61B 2562/0219

(57) ABSTRACT

Systems, device, and methods may be used to insert time notations into recording and/or signals such as video recordings, physiological signals for user, and/or recordings of video game play. The time notations may be added responsively to a user command, such as a verbal and/or gestural command received via a wearable portable device and/or activation of, for example, button of a video game controller and/or keyboard. Additionally, or alternatively, the time notations may be added to physiological signals for a user upon detection of an anomaly within the signal so that, for example, the portion of the signal that has the anomaly may be searched for at a later time for further consideration and/or analysis.

### RELATED APPLICATION

This application is a NON-PROVISIONAL of U.S. Provisional Patent Application No. 63/649,297 filed on 17 May 2024 and entitled “SYSTEMS, DEVICES, AND METHODS FOR RECORDING A TIME NOTATION FOR CORRELATION WITH A VIDEO AND/OR AUDIO RECORDING”, which is incorporated herein in its entirety.

### FIELD

The present invention pertains to making and/or recording time notations for events that take place within a recording of, for example, biometric data taken over time, a video recording, audio recording, and/or a live stream of audio and/or visual information.

### BACKGROUND

Recordings taken over time often capture a plurality of events. The longer the recording, the more difficult it is to locate a particular event within the recording, which is tedious and frustrating. For example, webcams posted at parks, beaches, or other venues often capture video and/or audio recordings twenty-four hours a day or during daylight hours for various purposes including, for example, monitoring the areas for wildlife activity or to provide the video to interested parties via website. For example, a webcam recording activity in the ocean proximate a beach may capture video all day long of surfers surfing in the ocean. If a surfer then wants to see the video of them surfing, they must access the webcam recording and manually search through the recording to try to locate the portion capturing when he or she was surfing at the beach, which can be frustrating and time consuming.

### SUMMARY

The systems, devices, and methods disclosed herein and, in particular, a processor of a portable device, may be configured to receive a command (e.g., voice command, gestural command, change in the user's geolocation, manual activation of a button (e.g., a button on a side of a watch and/or on a surface of a fob), manual selection of an icon, graphic, and/or text provided by a touch screen, and/or change in the user's physiological information) to record a time notation and may record the time notation responsively to the received command. In some instances, a user may provide an instruction regarding an event that may trigger issuing a command to record a time notation and, when the event occurs, the command to record a time notation may be received in accordance with the instruction.

The time notation may be communicated from the processor of a portable device to a processor of an external computing device, which may be configured to prepare a query for a database of recordings, the query including a request for a segment of a recording that is associated with the time notation. The segment of the recording associated with the time notation may be received responsively to the query and communicated to a display device.

In some embodiments, the portable device may be a wearable device worn by a user and may include a global positioning device (GPS) configured to determine a geolocation for the user. In these embodiments, the GPS may determine a geolocation of the portable device when the command is received and communicate that geolocation to, for example, the processor of the portable device so that the position with the time notation may be associated with one another in a memory in communication with the processor of the portable device. Additionally, or alternatively, the processor of the portable device may receive an instruction to record a time notation when the portable device is proximate to a geolocation. Geolocation information for the portable device may be received over time from the global positioning device and analyzed to determine whether the portable device is proximate to the geolocation and, if so, recording of a time notation may be triggered and/or performed.

Additionally, or alternatively, the portable device may be a wearable device worn by a user that includes a physiology sensor in communication with the processor of the portable device. The physiology sensor may be configured to obtain physiological data and/or a stream of physiological data over time (e.g., hours, days, etc.) for the user including, but not limited to, heartrate, pulse, calories burned, pulse oximetry information, EEG information, and/or electrocardiogram (ECG) information. When the physiological information for the user is received from the physiology sensor, it may be analyzed to determine physiological information for the user and, at times, a time notation may be associated with the physiological information responsively to, for example, an event, change, and/or a data anomaly (e.g., an elevated heartrate or low pulse oximetry values) so that, for example, the event and/or anomaly may later be searched for, and/or pinpointed within in the stream of physiological data.

In some embodiments, a physiological signal for a user may be received from a physiological monitor over time. The physiological signal may be analyzed for events included in the signal and, when detected, a segment of the physiological signal may be associated with a time notation to indicate when the event occurs in time. Then, the physiological signal and a timestamp corresponding to the event may be saved in a memory and/or database. Next, a request for the segment of the physiological signal that is associated with the timestamp may be received and the database may be queried for the segment using the time notation. The segment of the recording associated with the time notation may then be received responsively to the query and provided to a display device.

In some embodiments, the systems, devices, and methods disclosed herein may be used to monitor a heart rate for a user by receiving (e.g., by a processor) a heartrate signal for the user over time while the user is performing a task. The heartrate signal may be received from a heartrate sensor in communication with the processor, which may analyze the heartrate signal in, for example, real time or nearly real time for events and/or anomalies (e.g., elevated heart rate and/or spikes of increased heart rate) and, when detected, may trigger and/or initiate the operation of an additional device (other than the heartrate sensor) to, for example, gain more information about what may have caused the events and/or anomalies and/or mitigate an event that caused the anomaly. The additional device may be, for example, a sensor, an alarm, a light, and a device configured to communicate a message. In one example, the task may be driving a vehicle and the additional device may be a camera and a radar sensor configured to image an environment in which the user is driving to, for example, detect hazards and/or events. In another example, the task may be providing services in the sales, hospitality, and/or gaming industry and the additional device may be a camera and/or microphone configured to film and/or record what is happening proximate to the user. In another example, the task may be related to law enforcement and the additional device may be a camera, location sensor, and/or accelerometer configured to configured to film and/or record what is happening proximate to the user, determine the user's location, and/or determine whether the user is moving and, if so, how the user is moving.

In other embodiments, the systems, devices, and methods disclosed herein may be used to access a video game recording while a user is playing a video game and receive an instruction to associate a time notation with a segment of the video game recording from the user while the user is playing the video game. Then, the video game recording may be modified by inserting time notation into the video game recording and associating it with the segment responsively to the instruction. The modified video game recording may then be stored in, for example, a memory and/or database. In some instances, a request for the segment may be received. The request may include the time notation.

A query for the database for the segment using the time notation may then be generated and used to query the database. The queried—for segment may then be received and provided to a display device for viewing by the user.

In some embodiments, the instruction may be received from a video game controlling device operated by the user. In these embodiments, the instruction may be received from, for example, a button or key mapped to an instruction to insert a time notation into the video game recording. Additionally, or alternatively, an annotation for the segment may be received from the user. The annotation may include information regarding an event that occurs during the segment and may include, for example, narrative text, symbols, emojis, and/or graphic elements. Once received, the annotation may be associated with the video game recording and/or the time notation and the association(s) may be stored in the database.

### BRIEF DESCRIPTION OF DRAWINGS

The present invention is illustrated by way of example, and not limitation, in the figures of the accompanying drawings in which:

FIG. 1A provides a block diagram of an exemplary system configured to perform one or more of the methods, or method steps, disclosed herein, in accordance with some embodiments of the present invention.

FIG. 1B provides a block diagram of an exemplary device configured to perform one or more of the methods, or method steps, disclosed herein, in accordance with some embodiments of the present invention.

FIG. 2 is a flowchart illustrating an exemplary process for recording a time notation for correlation with a recording and/or locating a segment within a recording, in accordance with some embodiments of the present invention.

FIG. 3A is a graph showing the heart rate of a user over time, in accordance with some embodiments of the present invention.

FIG. 3B is a screen shot illustrating an exemplary graphic user interface (GUI), or dashboard, that may display information about various surf conditions to a user via a display device, in accordance with some embodiments of the present invention.

FIG. 4 is a flowchart illustrating an exemplary process for recording a time notation for correlation with a recording and providing the time notation to a user for locating a segment within a recording, in accordance with some embodiments of the present invention.

FIG. 5 is a screen shot illustrating an exemplary manner in which the process of FIG. 4 may be executed, in accordance with some embodiments of the present invention.

FIG. 6A is a diagram of portable device having a display device displaying a set of time notations for a video recording of a user riding a skateboard, in accordance with some embodiments of the present invention.

FIG. 6B is an image of a frame of a skateboarding video with a slider bar showing a plurality of images included in the skateboarding video as well as time notations, in accordance with some embodiments of the present invention.

FIG. 7 is a flowchart illustrating an exemplary process, in accordance with some embodiments of the present invention.

FIG. 8 is a screen shot provides of a GUI providing map and a plurality of routes between an beginning and ending point on the map, in accordance with some embodiments of the present invention.

Throughout the drawings, the same reference numerals, and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### Written Description

At times, contemporaneous notation and/or capture of time notations, or timestamps, that correspond to one or more events that may be captured by a recording (e.g., sound, video, series of images, etc.) and/or signal (e.g., a signal carrying physiological information) may be desired in order to, for example, efficiently locate and/or find a portion of the recording and/or signal (also referred to herein as a “recording segment”) that corresponds to the event at a later time. Disclosed herein are devices, systems, and methods for contemporaneously recording time notations so that a user may later use these time notations to find recording segments that correspond to the time notations. The systems, devices, and methods disclosed herein may be used when it is desirable to easily and conveniently note, or record, a time at which an event of interest (e.g., start, stop, a moment of particular interest, etc.) occurs (without disrupting the user's participation in the event) so that the user may later use the time notation to find a recording segment of the event of interest within the larger recording without having to view and/or process the entire recording.

The time notations disclosed herein may be recorded responsively to any type of command including, but not limited to, a voice command, a gestural command, biometric data, a change in biometric data, GPS coordinates, and/or a user input via an interface (e.g., GUI, button, or touch screen) with or without connectivity of Wi-Fi or BLUETOOTH™ to a mobile device, application, or third party application. Additionally, or alternatively, a time notation and/or timestamp may be recorded responsively to an event present within a physiological signal such as a change in heartrate, pulse, respiratory rate, electroencephalogram wave (EEG) patterns and/or information, and/or electrocardiogram (ECG) wave patterns and/or information.

The time notations may be stored in the memory of a wearable device worn by the user and/or communicated to an external device such as a cloud computing environment or computer. The methods disclosed herein may be executed by a dedicated device (e.g., a wearable device such as a watch, badge, and/or fob) and/or a software application running on a multi-purpose device (e.g., smart watch or smart phone). The dedicated device may be configured to pair and/or communicate via, for example, WIFI, BLUETOOTH™, etc.) with an external device like a computer, server, and/or cloud computing environment, to facilitate access to recordings of interest and/or segments of interest within a particular recording. In some embodiments, the recordings may be general recordings of a location (e.g., a beach, ocean, mountain, ski run, trail, area of sky, etc.) that may be recorded by a third party in which a user may appear when, for example, participating in a sport or activity and the time notations may assist the user in finding a segment of these general recordings in which they appear and/or are doing something of interest (e.g., riding a wave or skiing down a trail). Additionally, or alternatively, the recordings may be specific to a particular user and/or group of users that may be recorded by the user.

In some embodiments, the systems, devices, and/or methods disclosed herein may be used to add time notations to recordings of users playing video games. In these embodiments, a user and/or a third party may record video game play and the user may add time notations to the recording via, for example, a button on his/her/their video game controller and/or keyboard programmed to add time notations to a video game recording throughout the timeline of video game play to, for example, make note of events during video game play that may be of interest to the user and/or viewers of the video game recording. In some embodiments, the user may also add notes or other context to the time notations to, for example, add in a description of an event associated with a time notation. This may assist the user and/or other viewers of the recording in locating events of interest that occur during game play, which may increase the user's and/or viewers enjoyment of the video game recordings, engagement (e.g., length of time viewers watch the user's video game recordings) with viewers, and/or a number of viewers who watch the user's video game recordings, which may be linked to revenue gained when viewers watch the user's video game recordings.

In some embodiments, the systems, devices, and/or methods disclosed herein may be used to add time notations to recordings of EEG information recorded for a user in, for example, military and/or law enforcement contexts and/or to monitor a health and/or wellness of a user. In a military context, the systems, devices, and/or methods disclosed herein may be used to add time notations to recordings of a soldier's EEG information to annotate activity the soldier is participating in and/or monitor the soldier's brainwaves for signs of irregularities that may indicate, for example, when the soldier is engaged in combat and/or may be suffering from an adverse health outcomes due to a situation the soldier is involved in. Additionally, or alternatively, the systems, devices, and/or methods disclosed herein may be used to add time notations to recordings of a user's EEG information to annotate brain wave activity in response to stimulus provided by, for example, the activities of daily life including, but not limited to, walking, sleeping, watching a video, listening to music, using a computer, playing a game, playing a video game, and participating in a virtual reality activity, wherein time notations may be added to the EEG recording to note, for example, initiation of an activity, cessation of an activity, and/or an event that occurs during participation in the activity. Additionally, or alternatively, the systems, devices, and/or methods disclosed herein may be used to add time notations to recordings of a user's EEG information to annotate brain wave activity while the user is being monitored to assess his/her/their medical condition (e.g., a neurologic condition like epilepsy or Parkinson's Disease), during recovery from an injury and/or surgery. These time notations may then be used to find portions of EEG recordings that correspond to events that may be used to better understand the user's neurological responses to the events in these different contexts.

In some embodiments, the systems, devices, and methods disclosed herein may be used to record time notations with, for example, a backend time notation in a universal timekeeping format (e.g., coordinated universal time (UTC), International Organization for Standards (ISO) 8601, Portable Operating System Interface (POSIX) time, and/or Society of Motion Picture and Television Engineers (SMPTE) time, which is an electronic signal which is used to identify a precise location on time-based media such as audio or video tape or in digital systems. Additionally, or alternatively, the systems, devices, and methods disclosed herein may be used to record time notations with near-field communication protocols (e.g., BLUETOOTH™, Wi-Fi) social media logins, application programming interfaces (APIs), user profiles configured to enable lateral software application usage, and/or native device dashboards configured to import information from third parties to a centralized dashboard. Additionally, or alternatively, time notation recording as described herein may be integrated into software applications via, for example, the systems, devices, and methods disclosed herein.

Benefits of the systems, devices, and methods disclosed herein include, but are not limited to, cost savings and/or reducing energy consumption for processing video and other recordings because segments of interest within the recordings will be easier to locate within the recording using the time notations disclosed herein. This may be of particular impact for reducing input and/or output overhead when, for example, using machine learning to build, modify, and/or implement algorithms, training artificial intelligence models and/or performing inference on rolling windows of data like video and/or audio recordings. Additionally, or alternatively, benefits of the systems, devices, and methods disclosed herein may include improved sequential and/or temporal ordering of events by machine learning models, which may assist with more efficient and/or accurate retrieval of data from a database. For example, when a database storing video and/or audio recordings is queried for video segments of interest using time notations, the queried—for video segments may be returned from the database in, for example, a time-sequential order, with optional support for missing values or irregular time intervals. Additionally, or alternatively, benefits of the systems, devices, and methods disclosed herein may include enhanced scalability training of models and algorithms by performing multi-tier extract, transform, and load indexing across large datasets via, for example, a personal dashboard or network wide dashboard of datapoints using, for example, physiological and/or position data for a user that, for example, may be compiled throughout a software application running on a portable device. native app, third party IoT [Internet-of-Things] apps

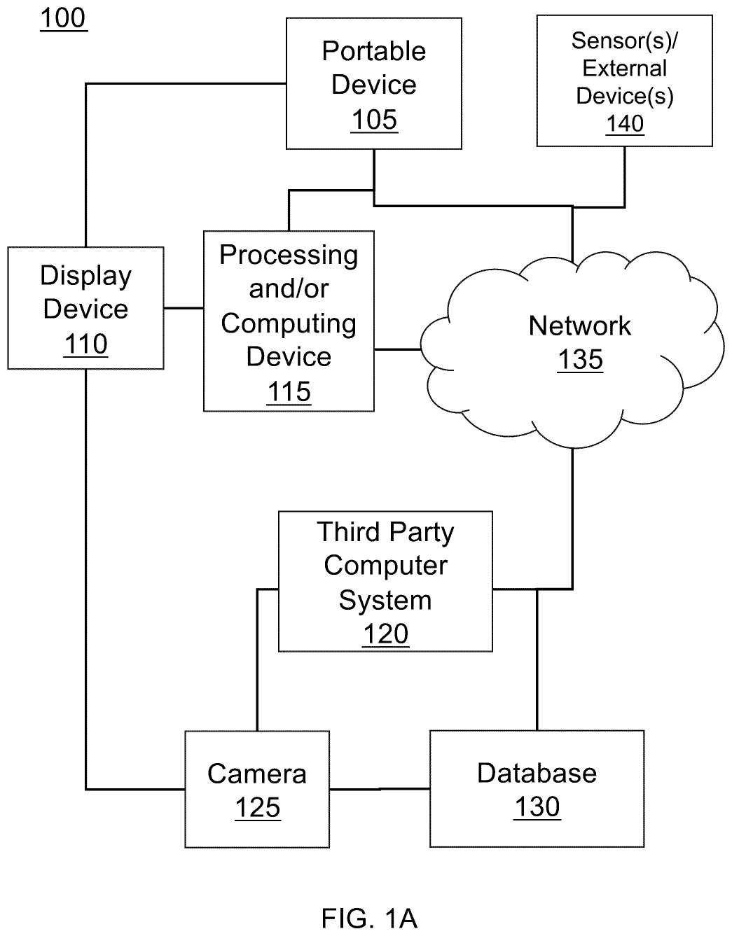

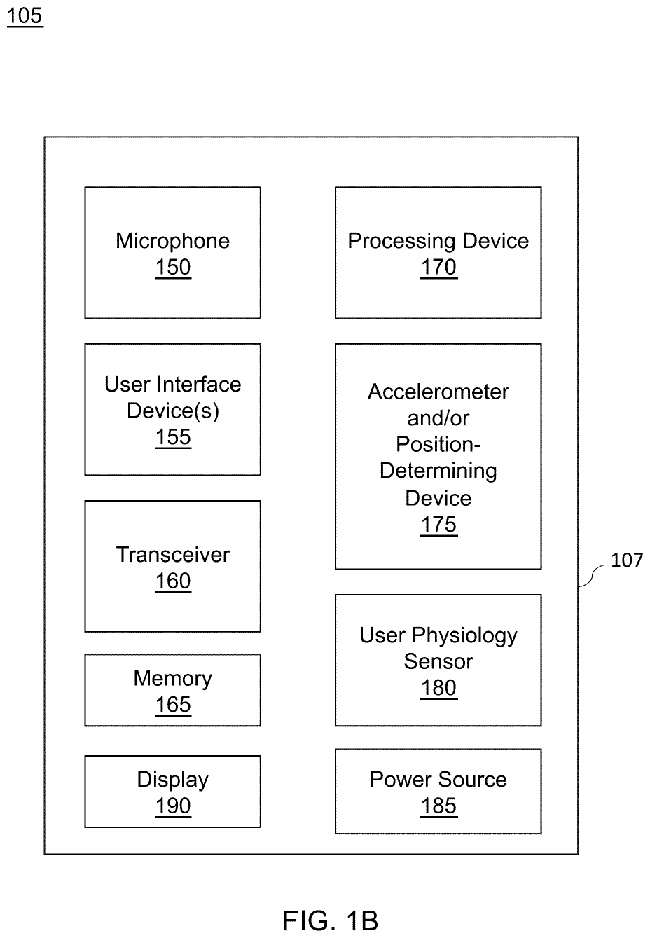

Turning now to the figures, FIG. 1A is a block diagram of an exemplary system 100 in which one or more steps of the methods disclosed herein may be executed. System 100 includes a portable device 105, a display device 110, a processing and/or computing device 115, an optional third party 120, a camera 125, a database 130, and one or more sensor(s) 140. One or more components of system 100 may be connected together via communication network 135, which may be embodied as the Internet and/or one or more wired and/or wireless connections that may be facilitated by one or more near-field communication, protocols, including, but not limited to, BLUETOOTH™. Communication between two or more components of system 100 and/or software programs running on one or more components of system 100 may be facilitated by one or more application programming interfaces (API).

Processing and/or computing device 115 may be any processing and/or computer device configured to execute one or more steps of the methods disclosed herein. In some embodiments, processing and/or computing device 115 may be resident within portable device 105. Additionally, or alternatively, processing and/or computing device 115 may be embodied as a device separate from portable device 105. Exemplary processing and/or computing devices include, but are not limited to, smart phones, wearable devices, tablet computers, laptop computers, and/or field programmable gate array (FPGA) devices and/or component of system 100, such as portable device 105, processing and/or computing device 115, third party computer system 120, camera 125, and/or database 130. Display device 110 may also be configured to display, or provide, auditory and/or video information (e.g., a video recording or video recording segment) to a user. Exemplary display devices 110 include, but are not limited to, video screens, computer display screens, touchscreens, speakers, and/or microphones.

Camera 125 may be any camera configured to record one or more events of interest as they occur over time. Camera 125 may be a still or video camera configured to record events on a continuous, as-needed, and periodic basis over time. In some embodiments, camera 125 may be resident within a moving device (e.g., a drone or satellite) and the moving device may be configured to, for example, follow a moving object or person and/or facilitate the imaging and/or videotaping of a geographical area (e.g., coastline or mountain) by camera 125 to, for example, record activity within the geographical area. Activity that may be recorded includes, but is not limited to, movement of people, animals, or features of the environment proximate to the geographical area (e.g., water, trees, snowfall, etc.). Camera 125 may be owned and/or operated by an entity providing portable device 105, a software program running on processing and/or computing device 115 that enables execution of one or more processes and/or processing steps, disclosed herein, and/or a third-party 120, such as a company (e.g., ski resort, equipment rental company, race track, etc.), park service, security agency, and/or governmental entity as a way of, for example, recording activity, or events, that occur in particular places of interest (e.g., action-sports locations (e.g., ski resorts, race tracks, skateboard parks, coastlines, etc.), building entrances, within an ambulance, etc.) or times of interest such as when an event occurs, when an event is expected to occur, when an event concludes, at dawn, at sunset, when a police officer enters a dwelling in search of a suspect, when a racer crosses a finish line, when a surfer dismounts his or her surfboard, etc. Database 130 may be configured to store one or more recordings captured by camera 125 and may be directly and/or indirectly accessible and/or searchable via processing and/or computing device 115 and/or third party 120.

Sensor(s) and/or external device 140 may be one or more additional sensors that may, or may not, be included in portable device 105. Exemplary sensors 140 include, but are not limited to, cameras, radar, microphones, and sensors on a vehicle to configured to trigger, for example, automatic braking. Exemplary external devices include, but are not limited to, communication devices (e.g., walkie-talkies, phones, etc.), lights, in some embodiments, one or more sensors 140 may be activated responsively to detection of an event within, for example, a signal carrying physiological information for a user. For example, if portable device 105 and/or another device in communication with portable device 105 is configured to monitor a user's heartrate, additional sensors may be activated when, for example, rapid increase in heartrate occurs. This may be useful in, for example, a law enforcement context, wherein when an elevation in a police officer's heartrate is detected additional sensors in the form of a body camera and/or microphone may be turned on. In another example, when an elevation in a commercial truck driver's heartrate is detected additional sensors in the form of radar and/or cameras designed to image other vehicles proximate to the driver's vehicle and/or microphone may be turned on to, for example, record events (e.g., another car cutting off coming too close to the truck or the truck driver missing a turn on a route) that may have caused the truck driver's heartrate to increase.

FIG. 1B is a block diagram of exemplary components that may be included in portable device 105, such as a microphone 150, one or more user interface device(s) 155, a transceiver 160, a memory 165, a processing device 170, an optional accelerometer and/or position-determining device 175, an optional user physiology sensor 180, a power source 185, and a display 190 (e.g., LED display or touch screen) that may be resident within a housing 107. Housing 107 may be configured as a water- and/or impact-resistant wearable device (e.g., watch, fob to hang from a bracelet or necklace, etc.). User interface device(s) 155 may be configured to enable a user to interact with (e.g., provide commands to and/or receive information from) portable device 105. Exemplary user interface devices 155 include, but are not limited to, display screens, touchscreens, lights, speakers, and keyboards. Accelerometer and/or position-determining device 175 may be any device, or set of devices, configured to determine a position of, and/or movement by, the user including, but not limited to, a global positioning system (GPS) device, a pedometer, a gyroscope, a velocity meter, and a two- and/or three-dimensional accelerometer. User physiology sensor 180 may be any sensor, or set of sensors, configured to measure one or more physiological parameters for the user. Exemplary physiological parameters include, but are not limited to, heartrate, pulse, calories burned, pulse oximetry information, EEG information, and ECG information. Power source 185 may be embodied as, for example, a rechargeable or disposable battery.

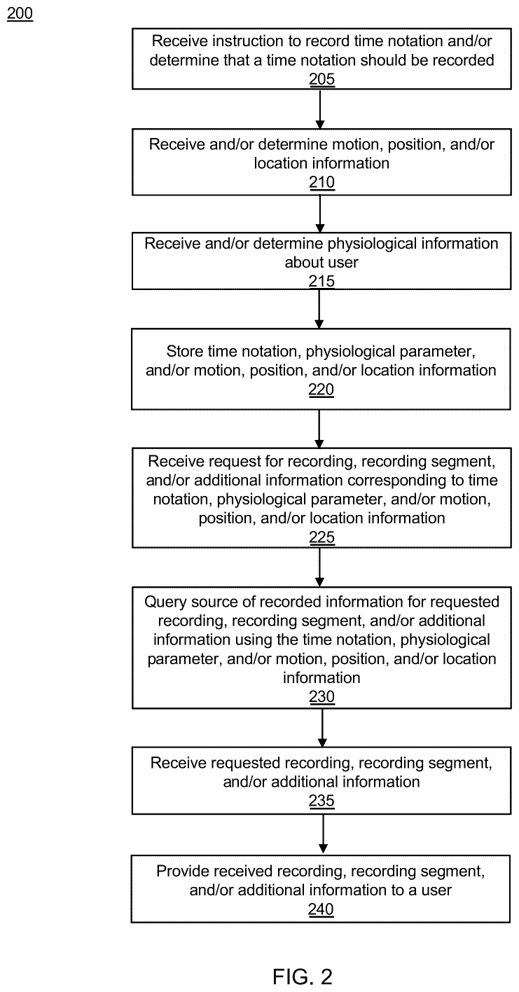

Memory 165 may be configured to store one or more sets of instructions (e.g., instructions for the execution of one or more steps of process 200 of FIG. 2, described below) that are executable by processing device 170. For example, memory 165 may be configured to store one or more time notations that may be reviewed, saved, and/or deleted via user interaction with a user interface device (155) such as a touch screen that displays icons or other GUI elements configured to allow the user to toggle through one or more time notations and so that they may be, for example, saved, deleted, and/or downloaded to, for example, processing and/or computing device 115. Additionally, or alternatively, memory 165, may be configured to store one or more user instructions, and/or commands that may be received via, for example, microphone, 150 and/or user interface devices 155. Processing device 170 may be any device configured to execute one or more instructions provided by a memory 165 and/or enable one or more operations of portable delays 105. Exemplary processing devices 170 include, but are not moved to, FPGAs, ASICs, and CPUs.

Transceiver 160 may enable communication between portable device 105 and an external device, such as processing and/or computing device 115 according to, for example, one or more instructions from processing device 170. Transceiver 160 may be embodied as, for example, an antenna, a transmitter, a wireless, communication port, and/or a wired communication port. In some embodiments, one or more components of portable device 105 such as microphone 150, user interface device(s) 155, transceiver 160, accelerometer, user physiology sensor 180, and/or position determining device 175, and/or processing device 170 may be configured to detect and/or respond to emergency situations such as evidence of a sudden stop that may indicate a fall or crash, evidence that the user has moved to an unexpected location (e.g., fallen off a ski trail or has entered a dwelling). Additionally, or alternatively, a user may trigger an emergency situation response by portable device 105 by, for example, speaking a code word (e.g., “help” or “emergency”) or by manually triggering an alarm via, for example, interaction with user interface device(s) 155. When an emergency situation is detected and/or triggered, one or more components of portable device 105 may communicate an indication of the emergency situation. For example, upon detection of a crash, transceiver 160 may communicate an emergency alert to local authorities and/or a preferred contact of the user. Additionally, or alternatively, upon detection of an emergency situation, user interface device(s) 155 may emit an audible sound and/or light flashes.

FIG. 2 is a flowchart illustrating an exemplary process 200 for generating a time notation and/or timestamp for when an event occurs in a recording and associating the time notation and/or timestamp with the recording so that, for example, the time notation may later be used to efficiently find and/or access a segment of the recording associated with the event's occurrence. Process 200 may be executed by, for example, system 100 and/or a component thereof (e.g., portable device 105 and/or processing and/or computing device 115).

Initially, in step 205, an instruction to record, or store, a time notation for a recording may be received by, for example, a portable device like portable device 105 and/or a software program running on the portable device. In some embodiments, the instruction may be provided via user interaction with the portable device via, for example, a user speaking a verbal, or voice, command that is captured via microphone 150, a user tapping housing 107, and/or a user moving housing 107 in a specified (e.g., a gesture) manner. For example, portable device 105 may be configured to record a time notation when the user rapidly shakes portable device 105 three or four times or draws a “Z” on a touch screen of portable device 105. Additionally, or alternatively, the instruction may be received responsively to an event that is detected by, for example, the portable device and/or a device in communication with the portable device (e.g., a smart phone). For example, if the portable device includes a heartrate monitor (e.g., pulse meter, ECG, etc.) and a change in the user's heart rate it detected, step 205 may be executed so that a timestamp is associated with the recording at a time that corresponds to the event. In another example, if the portable device includes an accelerometer, step 205 may be executed when a change in the user's acceleration and/or position Is detected so that a timestamp is associated with the recording at a time that corresponds to the event.

Optionally, portable device 105 may further receive and/or determine motion, position, and/or location information for the user (step 210). The position, motion, and/or location information may be received from, for example, accelerometer and/or position-determining device 175. In some embodiments, execution of step 210 includes detecting motion (e.g., standing up, moving forward or backward, etc.), linear and/or angular acceleration, and/or determining a position of the user and/or portable device 105.

Optionally, portable device 105 may further receive and/or determine physiological information, a change in physiological information (e.g., an elevated heart rate or irregular ECG heart rate pattern), variations in EEG patterns, and/or more physiological parameters for the user (step 215). The physiological information and/or parameters may be received from, for example, user physiology sensor 180. In step 220, the time notation, physiological information, motion information, position information, and/or location information may be stored on, for example, memory 165 and/or communicated to an external device (e.g., processing and/or computing device 115) by transceiver 160.

In some embodiments, execution of step 205 may be responsive to the motion information, position information, location information, physiological information, and/or physiological parameter received and/or determined via execution of steps 210 and/or 215, respectively and/or a change in the motion information, position information, location information, physiological information, and/or physiological parameter. In these embodiments, a time-notation command may be received responsively to a determination that a preset threshold and/or value for the motion information, position information, location information, and/or a physiological information has been met, or exceeded. For example, in a surfing context, when portable device 105 registers, or detects, motion consistent with beginning to surf, or ride, a wave (e.g., transitioning from sitting on a surfboard to standing on it, or acceleration at a rate greater than what is experienced by sitting on a surfboard in the ocean), the time at which this motion is detected may be recorded and, at times, a time at which motion consistent with concluding the riding of the wave (e.g., sitting down, deceleration, etc.) is detected may also be recorded. In another example, in a skiing context, when a sound consistent with a skier passing through a starting gate is heard by, for example, a microphone of a portable device like portable device 105, a time notation for time at which the sound occurs and/or is detected may be recorded. Continuing with the skiing example, when a sound consistent with being aloft (i.e., not in contact with the ground) is heard and/or an absence of sound consistent with skiing along the ground is detected, a time notation for time at which this occurs may be recorded. In another example that may be executed in a law enforcement context, a time notation may be recorded when, for example, a police officer exits his or her vehicle (which may be determined via, for example, detecting that the police officer has stood up and/or a sound consistent with the closing of a vehicle door is detected by the portable device and/or another device), enters a building, announces himself or herself as a police officer, and/or begins to run.

In another example, a user and/or third party may record video game play and use portable device 105 and/or a button on a video game controller and/or keyboard to make time notations for events or segments of a video game recording that may be of interest. These time notations may later be used to find segments of a video game recording that are of interest. In some embodiments, steps 210, 215, and/or 220 may be run as background processes that receive and/or determine motion, position, location, and/or user physiological information on a continuous, periodic, or as-needed basis regardless of user input. When a button on a video game controller and/or keyboard it used in embodiments executing this example, the button may be remapped according to, for example, parameters for the video game controller, keyboard, and/or game being played. In some embodiments, the remapping may be basic remapping performed at the in-game and/or system level(s) by changing a button mapped to an action (e.g., jump and/or shoot) to be mapped to capturing, recording, and/or making a time notation. Additionally, or alternatively, remapping may be done at the system and/or operating system level by mapping buttons to keyboard/mouse inputs, creating macros, and/or profiles to capture and/or make a time notation upon activation (e.g., the user pressing the button or otherwise entering a command via his/her/their platform and/or pressing a key, or combination of keys, to trigger the macro. Additionally, or alternatively, remapping may be done by remapping hardware (e.g., an adaptive controller) to physically remap switches, buttons, or paddles or software to capture, record, and/or make a time notation.

In step 225, a request for a recording and/or recording segment corresponding to the time notation, physiological information and/or parameter, motion information, position information, and/or location information may be received from, for example, the user via the user's interaction (e.g., a voice command, interaction with a GUI of portable device 105, and/or interaction with a user interface of portable device 105) with a software application running on portable device 105 and/or processing and/or computing device 115. Additionally, or alternatively, the software application may enable communication between portable device 105 and third party 120 and/or processing and/or computing device 115 to retrieve a recording segment corresponding to one or more recorded time notations. The request of step 225 may include the time notation, physiological information, physiological parameter, motion information, position information, and/or location information of steps 205, 210, and/or 215, which may, for example, be communicated by transceiver 160 to processing and/or computing device 115.

In some embodiments, the request of step 225 may include a request for additional information that may be associated with, and/or correlated to, one or more time notation(s) and/or recordings. Exemplary additional information that may be included in the request of step 225 includes, but is not limited to, the user's heart rate, the user's EEG information, the user's ECG information, the user's location, the user's velocity, the user's acceleration, the user's position, the user's height above sea level, atmospheric conditions proximate to the user, environmental conditions proximate to the user, terrain conditions proximate to the user, ocean conditions proximate to the user, a video game the user is playing, a type of controller the user is using to play a video game, a platform the user is using to play a video game, a service the user is using to record playing a video game, and how a user is recording a time notation (e.g., manual activation of a button on a video game controller that may be mapped to execution of recording a time notation and/or interaction with an interface used to play the video game) while playing a video game.

In step 230, a query for the requested recording, recording segment, and/or additional information may then be prepared and communicated to a source of the recording (e.g., third party 120, camera 125, and/or database 130) by, for example, the software application responsively to the request of step 225. The requested recording, recording segment, and/or additional information may then be received by, for example, the software application (step 235) and provided to the user (step 240) via, for example, provision of the recording or recording segment to display device 110. In some embodiments, additional information may be provided along with the recording and/or recording segment so that the additional information may be presented in context with the recording and/or recording segment. Continuing with the surfing example above, a video recording segment showing the user when riding a wave may be provided to the user along with additional information such as wave height, wave velocity, weather conditions, geolocation information, heart rate while surfing, and/or average number of calories burned during the recording segment.

In one use case, process 200 (or portions thereof) may be executed by a surfer surfing in the ocean when the beach and ocean proximate to the surfer are being recorded over time by, for example, a third party service and/or webcam that may not be operated by the surfer (e.g., a webcam that records the beach and ocean 24 hours a day). When the surfer begins to surf a particular wave, an event may occur (e.g., acceleration or change in position that may be consistent with riding a wave) and/or the surfer may provide a command (e.g., touch a button or touch screen, turn a dial, make a gesture, speak a verbal command, etc.) to portable device 105 (step 205) and the portable device may record a notation of the time at which the command was provided by the surfer (step 220). Then, at some later time, the surfer may access a recording of the ocean where he or she surfed and may use the recorded time notation to pinpoint a recording segment within the larger recording during which he or she was surfing the wave. This saves the surfer the time and energy typically required to watch the whole recording to find the recording segment of interest (i.e., when he or she was riding the wave) and greatly increases the efficiency with which the surfer can find recording segments correlated to events of interest to him/her.

In another use case, portable device 105 may be used to wirelessly communicate (e.g., BLUETOOTH™ or Wi-Fi) with another device (e.g., drone, smart glasses, body camera, video camera, etc.) to, for example, trigger an operation (e.g., turning on or off) of the device. For example, a user of portable device 105 and a drone that is communicatively coupled to portable device 105 may provide a command (e.g., verbal cue, gesture, etc.) to portable device that 105 that is communicated to the drone to trigger an operation of the drone such as audio-visual recording or adding a time notation to a recording.

On some occasions, biometric events may trigger time notations that can connectivity pair with mobile devices, applications, and other third party applications to pinpoint a recording(s) segment/point of interest when, for example, monitoring a patient in a hospital room, prisoner/detainee in a prison cell, a police officer on duty, and/or an individual participating in an online workout class.

In another example, physiological data for a user may be received by portable device 105 and portable device 105 may trigger an operation of another device (e.g., camera or audio recording) responsively to the physiological data. Continuing with this example, certain physiological data (e.g., elevated heart rate, elevated respiratory rate) that may be measured and/or received by portable device 105 may trigger a camera to turn on to capture audio-visual information proximate to the user as may be the case when, for example, a user wearing portable device 105 and a body camera is riding a roller coaster and the user's increased heart rate triggers recording of the body camera so that the user's experience of riding the roller coaster is captured by the body camera.

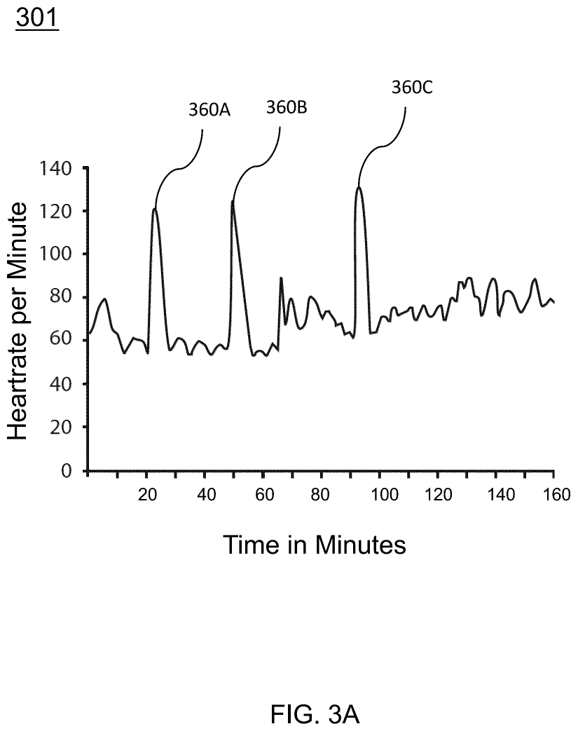

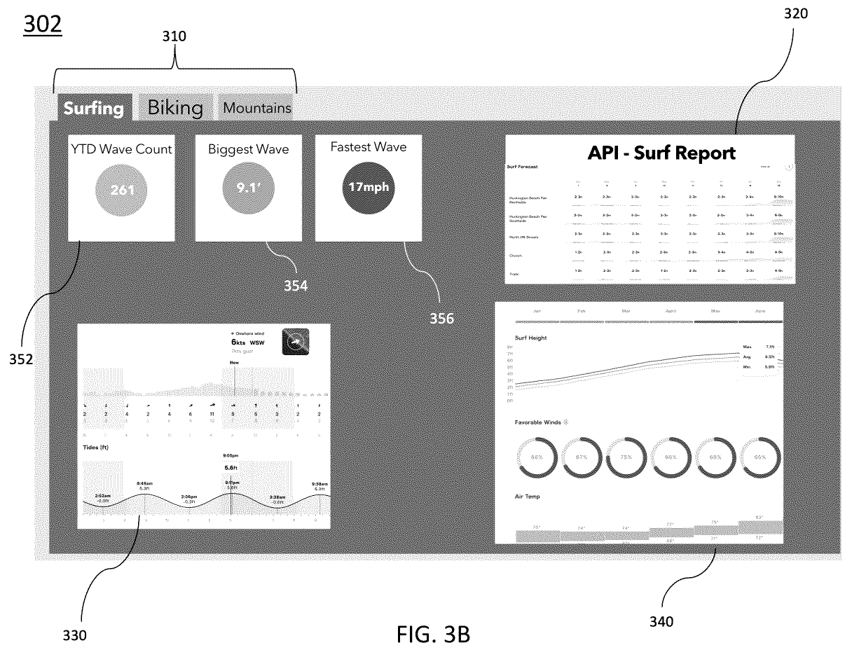

FIG. 3A provides a graph 301 plotting a user's heartrate per minute as a function of time in minutes over a time interval of 160 minutes. During the 160 minute interval, the user's heartrate stays within the 60-100 beats per minute, which is considered “normal” for most of the time but has sudden peaks when a first event 360A occurs at approximately 23 minutes, a second event 360B occurs at approximately 50 minutes, and a third event 360C occurs at approximately 93 minutes. For each of these events, a corresponding time notation may be recorded and/or a secondary action may be taken (e.g., turning on a sensor).

FIG. 3B is a screen shot illustrating an exemplary GUI, or dashboard, 302 that provides a plurality of windows that show information about various oceanic, weather, and surfing conditions that may be displayed via a display device like user interface 110. For example, dashboard 302 includes a set 310 of menu options (in this case, surfing, biking, and mountains) wherein the surfing option has been selected. Dashboard 302 also includes a first window 320 that provides a surf report, or surf forecast, for various locations, a second window 330 that provides information regarding wind and tides, and a third window 340 that provides information regarding surf height, percentage of the time of favorable winds, and air temperature. In addition, dashboard 302 provides a year to date wave count window 352, a biggest wave measurement 354, and a velocity for a fasted wave 356.

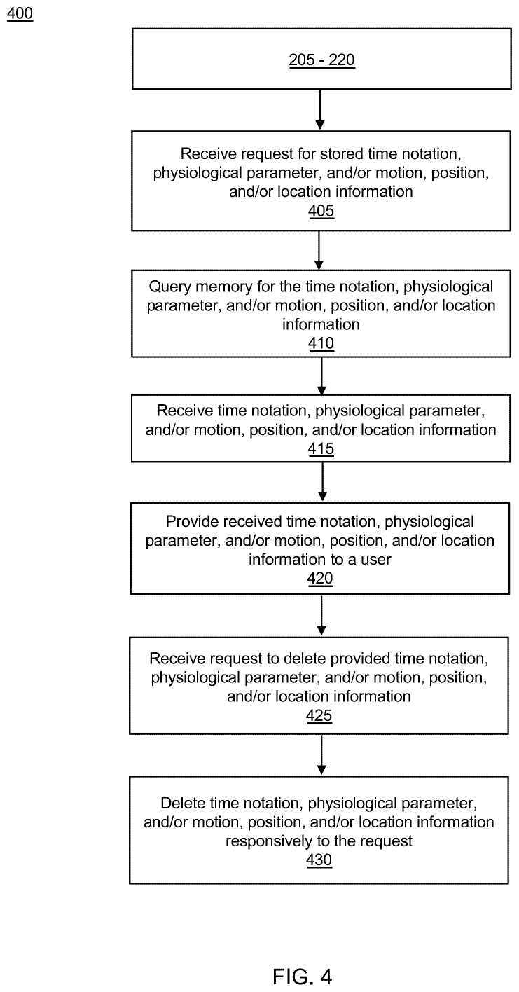

FIG. 4 is a flowchart illustrating an exemplary process 400 for recording a time notation for correlation with a recording and providing the time notation to a user for locating a segment within a recording, in accordance with some embodiments of the present invention. Process 400 may be executed by, for example, system 100 and/or a component thereof (e.g., portable device 105 and/or processing and/or computing device 115).

Initially, steps 205-220 may be executed as, for example, described above. Then, in step 405, a request for a stored time notation, physiological information physiological parameter, and/or motion, position, and/or location information for a user may be received via, for example, interaction with a software application running on portable device 105 and/or processing and/or computing device 115. The request of step 405 may be received via, for example, a voice, command/or interaction with a graphic user interface provided by portable device 105 and/or processing, and/or computing device 115.

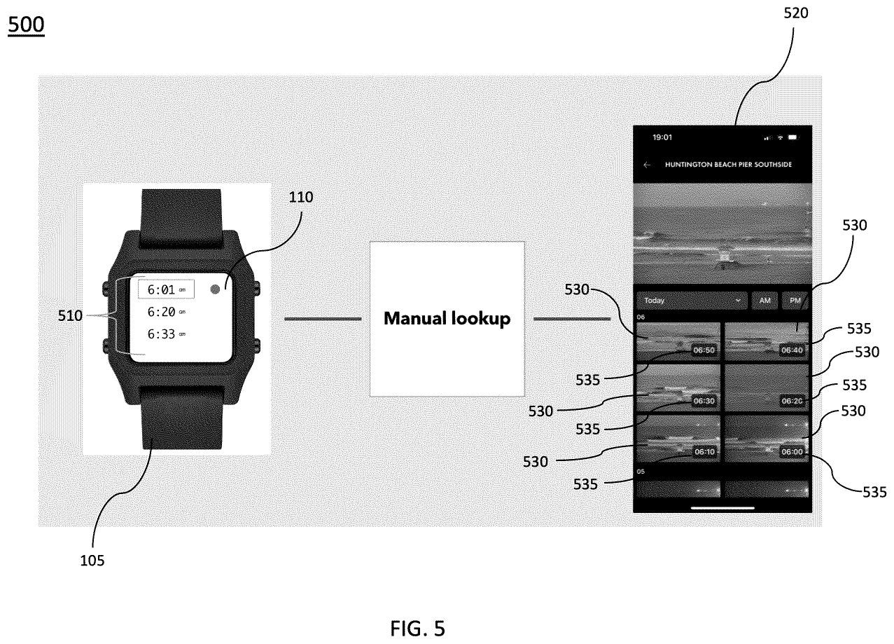

Next, in step 410, a memory such as memory 165 and/or a memory of processing and/or computing device 115 may be queried by, for example, processing device 170, for the requested time notation, physiological information, physiological parameter, and/or motion, position, and/or location information for the user and the queried for information may be received (step 415) and provided to the user (step 420). Step 420 may be executed by, for example, providing a visual display on a display device like display device 190. For example, as shown in FIG. 5, display 190 of portable device 105 provides a list 510 of stored time notations that have been provided via execution of process 400. The user may then manually access a source of video recordings (e.g., third party 120 and/or processing and/or computing device 115) to look up recording segments as shown in exemplary window 520, which includes an array (in this case, six) of videos 530 that have a different start times as shown in a start time window 535 associated with the time notations. The user may then use the time notations provided in list 510 to find a video of interest (using the start time window) and then find a segment within the video of interest.

Optionally, in step 425, a request to delete the time notation, physiological, parameter, and/or motion, position, and/or location information for the user provided in step 420 from memory 165 of portable device 105 of may be received and the provided time notation, physiological, parameter, and/or motion, position, and/or location information for the user may then be deleted from memory 165 responsively to the request (step 430). In some embodiments, execution of steps 425 and 430 may be part of a default routine and/or set of instructions configured to maximize available storage space for memory 165.

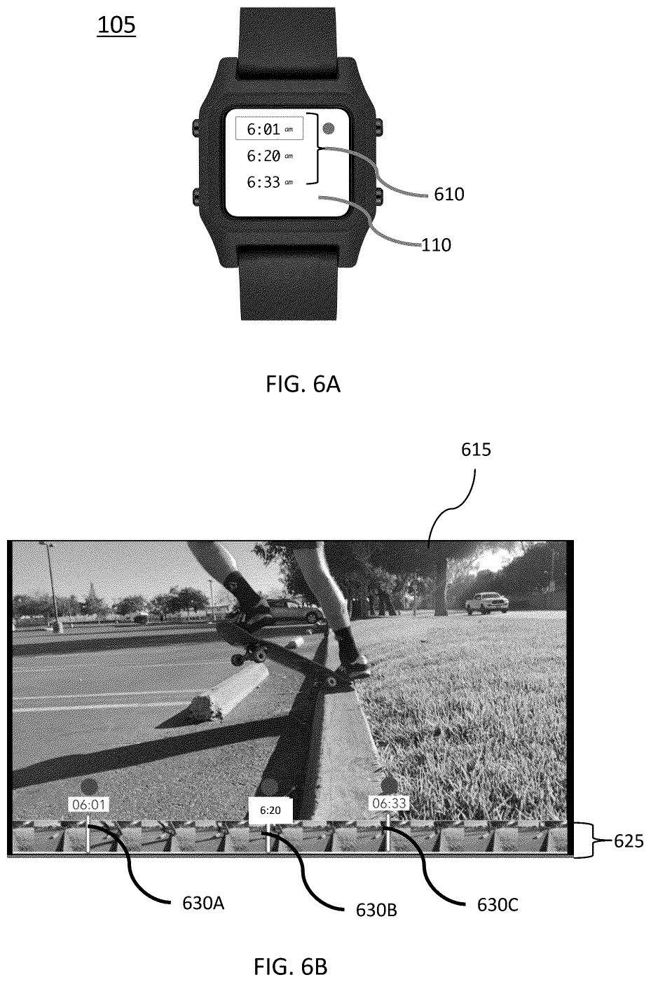

FIG. 6A is a diagram of portable device 105 having a display device 110 displaying a set of time notations 610 for a video recording of a user riding a skate board. FIG. 6B is an image 615 of a frame of the skateboarding video with a slider 625 showing a plurality of images included in the skateboarding video. FIG. 6B also shows a first, second, and third time notations 630A, 630B, and 630C that occur at times 06:01, 06:20, and 06:33, respectively, within the video. First, second, and third time notations 630A, 630B, and 630C, may be associated with and/or inserted into the skateboarding video as, for example, described herein and correspond to the first, second, and third time notations of set of time notations 610 shown on display 110.

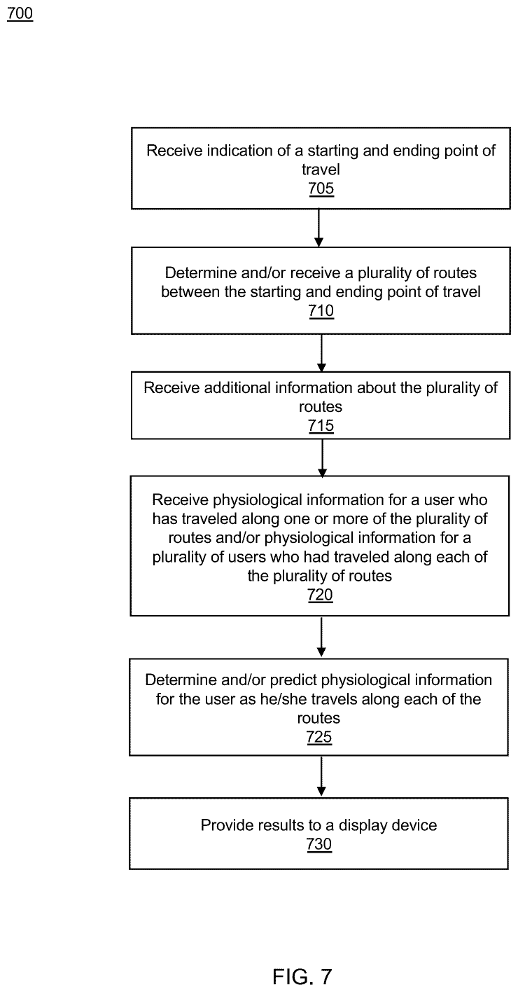

FIG. 7 is a flowchart illustrating an exemplary process 700 for providing physiological information about a user and/or a set of prior users as they traverse a route, or travel path, from a first point to a second point in space and/or on a map and/or recommend a path between two or more points based on a user preference for how the route may impact his/her physiology (e.g., heart beats per minute, pulse rate, respiration rate, etc.). Process 700 may be executed by, for example, system 100 and/or a component thereof (e.g., portable device 105 and/or processing and/or computing device 115).

In step 705, an indication of a starting point and an ending point of travel for a user and, optionally, a method of travel (e.g., walking, running, cycling, driving, etc.) may be received. Then, in step 710, a plurality of routes (e.g., 2-50) between the starting point and the ending point may be determined and/or retrieved from, for example, a map or other database such as database 130. Optionally, in step 715, additional information about the routes of step 710 may be received. The additionally information about the routes may include, but is not limited to, turns along the route, distance for the whole route and/or a portion thereof, elevation, changes in elevation along the route, curves along the route, and/or obstacles within the route.

In step 720, physiological information from the user and/or plurality of other users as they have traveled along one or more of a plurality of routes in the past may be received from, for example, a portable device like portable device 105 and/or a database like database 130. The physiological information may be, for example, an average heart rate of the user and/or an average heart rate for each user of the set of users as they travel along the respective plurality of routes for the entirety of the route or a set of route segments. Optionally, in step 725, the physiological information of step 720 and information about the corresponding route of step 710 and/or 715 may be used to determine and/or predict physiological aspects of each of the routes for the user. In step 730, a result of execution of steps 710, 715, 720, and/or 725 may be provided to the user on a display device like display device 110 and/or a display of portable device 105 so that, for example, the user may select the route that best matches their preferred physiological response (e.g., lower heart beats per minute and/or time spent) to traveling the selected route.

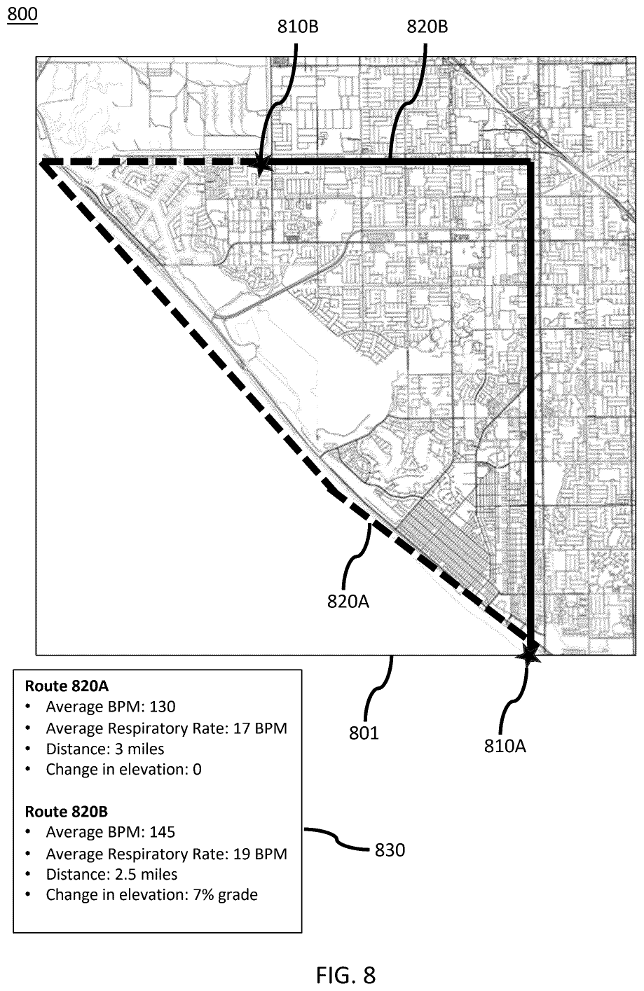

An example of how step 730 may be executed is provided by FIG. 8, which provides a GUI 800 showing map 801 of a portion of Los Angeles, CA proximate to the town of Huntington Beach with a starting point 810A and an ending point 810B and two routes in between. Starting point 810A and ending point 810B may be received from the user along with a mode of travel between the two routes (in this case, running). GUI 800 also includes a key 830 listing different information about a first route 820A between starting point 810A and ending point 810B, which is represented with a broken line and a second route 820B between starting point 810A and ending point 810B, which is represented with a solid line as well as previously received physiological information for the user when he/she has traveled along each of the routes. As indicated by key 830, information about first route 820A includes physiological information for the user when he or she traveled along the route in the form of an average heart rate/beats per minute (BPM) of 130, an average respiratory rate of 17 breaths per minute (BPM), and information about first route 820A in the form of a total distance of 3 miles with no change in elevation. As also indicated by key 830, information about second route 820B includes physiological information for the user when he or she traveled along the route in the form of an average heart rate/beats per minute (BPM) of 145, an average respiratory rate of 19 BPM, and information about second route 820B in the form of a total distance of 2.5 miles with a 7% grade change in elevation. Using this information, the user may select his or her preferred route (first route 820A or second route 820B) based on how rigorous a workout the user desires when running along the selected route.

I claim:

1. A method comprising:
receiving, by a processor of a portable device, a command to record a time notation;
recording, by the processor of the portable device, the time notation responsively to the received command;
communicating, by the portable device, the time notation to a processor of an external computing device;
preparing, by the processor, a query for a database of recordings, the query including a request for a segment of a recording that is associated with the time notation;
receiving, by the processor, the segment of the recording associated with the time notation responsively to the query; and
providing, by the processor, the segment of the recording to a display device.

2. The method of claim 1, wherein the portable device is a wearable device worn by a user and includes a global positioning device configured to determine a geolocation for the user, the method further comprising:
determining, by the global positioning device, a geolocation of the portable device when the command is received;
communicating, by the global positioning device, the geolocation to the processor of the portable device; and
associating, by the processor of the portable device, the geolocation with the time notation.

3. The method of claim 1, wherein the portable device is a wearable device worn by a user and includes a global positioning device configured to determine a geolocation for the user, the method further comprising:
receiving, by the processor of the portable device, an instruction to record a time notation when the portable device is proximate to a geolocation;
receiving, by the processor of the portable device, geolocation information for the portable device over time from the global positioning device;
analyzing, by the processor of the portable device, the geolocation information; and
determining, by the processor of the portable device, whether the portable device is proximate to the geolocation responsively to the analysis; and
triggering, by the processor of the portable device, recording of a time notation responsively to a determination that the portable is proximate to the geolocation.

4. The method of claim 1, wherein the command is at least one of a voice command, a gestural command, manual activation of a button, and manual selection of an icon provided by a touch screen.

5. The method of claim 1, wherein the portable device is a wearable device worn by a user and includes a physiology sensor in communication with the processor of the portable device, the physiology sensor being configured to obtain physiological data for the user, the method further comprising:
receiving, by the processor of the portable device, physiological information for the user from the physiology sensor;
analyzing, by the processor of the portable device, the physiological data to determine physiological information for the user; and
associating, by the processor of the portable device, the physiological information with the time notation responsively to the analysis.

6. The method of claim 5, wherein the physiological information is at least one of heartrate, pulse, calories burned, pulse oximetry information, EEG information, and electrocardiogram (ECG) information.

7. The method of claim 5, wherein the physiological information is a change in at least one of heartrate, pulse, calories burned, pulse oximetry information, EEG information, and electrocardiogram (ECG) information.

8. The method of claim 5, wherein the command is responsive to a change in user's physiological information.

9. The method of claim 1, further comprising:
receiving, by the portable device, an instruction from a user regarding a parameter for an event that automatically triggers issuing a command to record a time notation prior to receipt of a command to record a time notation, wherein when the event occurs, the command to record a time notation is received in accordance with the instruction.

10. The method of claim 1, wherein the portable device further includes an accelerometer configured to detect motion of the user and the command is a change in the user's acceleration or position.

11. The method of claim 1, wherein the portable device further includes an altimeter configured to detect an altitude of the user and the command is a change in the user's altitude.

12. A method for monitoring a heart rate for a user, the method comprising:
receiving, by a processor, a heartrate signal of a user over time while the user is performing a task from a heartrate sensor in communication with the processor;
analyzing, by a processor, the heartrate signal; and
triggering, by the processor, initiation of operation of a sensor other than the heartrate sensor upon detecting a change in the heartrate signal.

13. The method of claim 12, wherein the task is driving a vehicle and the sensor is at least one of a camera and a radar sensor.

14. The method of claim 12, wherein the task is providing services in at least one of a hospitality industry and a gaming industry and the sensor is a camera.

15. The method of claim 12, wherein the task is related to law enforcement and the sensor is at least one of a camera, a location sensor, and an accelerometer.

16. A computer-implemented method comprising:
accessing a video game recording while a user is playing a video game;
receiving an instruction to associate a time notation with a segment of the video game recording;
modifying the video game recording by inserting time notation into the video game recording and associating it with the segment responsively to the instruction; and
storing the modified video game recording in a database.

17. The computer-implemented method of claim 16, further comprising:
receiving a request for the segment, the request including the time notation;
generating a query for the database for the segment using the time notation;
querying the database using the query;
receiving the segment responsively to the querying; and
providing the segment to a display device.

18. The computer-implemented method of claim 16, wherein the instruction is received from a video game controlling device operated by the user.

19. The computer-implemented method of claim 16, further comprising:
receiving an annotation for the segment from the user, the annotation including information regarding an event that occurs during the segment; and
associating the annotation with the video game recording.
